(19) United States
(12) Patent Application Publication
(10) Pub. No.: US 2010/0234308 A1
(43) Pub. Date: Sep. 16, 2010

(54) WAKE-UP REMEDY

(75) Inventors: Miho Komatsu, Tsukuba-shi (JP); Koji Morishita, Tsukuba-shi (JP); Shin-ichi Hashimoto, Hofu-shi (JP)

Correspondence Address: DAY PITNEY LLP, 7 TIMES SQUARE, NEW YORK, NY, 10036-7311, US

(73) Assignee: Kyowa Hakko Kogyo Co., Ltd., Chiyoda-ku, Tokyo (JP)

(21) Appl. No.: 12/293,617

(22) PCT Filed: Mar. 23, 2007
(86) PCT No.: PCT/JP2007/055975

§ 371 Date: Oct. 6, 2008

(30) Foreign Application Priority Data
Mar. 23, 2006 (JP) 2006-080971

(51) Int. Cl. A61K 38/05; C07C 55/02; A61P 43/00

(52) U.S. Cl. 514/19; 562/563

(57) ABSTRACT

A wake-up remedy that for persons with the subjective symptoms of feeling languid after wake-up, having a hard time awaking, etc., relieves the symptoms and allows them to have a fulfilling life. There is provided a wake-up remedy containing alanylglutamine or its salt as an active ingredient.

### CROSS REFERENCE TO PRIOR RELATED APPLICATIONS

This application is a United States national phase application under 35 U.S.C. §371 of International Patent Application No. PCT/JP2007/055975, filed on Mar. 23, 2007, and claims the benefit of Japanese Patent Application No. 2006-080971, filed on Mar. 23, 2006, both of which are incorporated by reference herein. The International Application was published in Japanese on Oct. 25, 2007, as International Publication No. WO 2007/119503 A1 under PCT Article 21 (2).

### FIELD OF THE INVENTION

The present invention relates to a wake-up remedy containing alanylglutamine or a salt thereof as an active ingredient.

### BACKGROUND OF THE INVENTION

Alanylglutamine is a dipeptide containing two amino acids, alanine and glutamine, and is immediately degraded into alanine and glutamine in the body (refer to “Clinical Science”, 1988, Vol. 75, No. 5, p. 463-8). The action of glutamine is known to have many effects on physiological functions such as the regulation of skeletal muscle protein metabolism, repair of small intestine mucosa, and improvement of immunofunction, and it has been reported that the effects of alanine on physiological functions include an action to suppress blood sugar levels in diabetes patients (refer to “L-Alanyl-L-Glutamine”, Kyowa Hakko Co., Ltd., 2006, p. 1).

Alanylglutamine is superior in heat stability and solubility in aqueous solutions compared to glutamine, which has low-solubility and poor stability (refer to “L-Alanyl-L-Glutamine”, Kyowa Hakko Co., Ltd., 2006, p. 3), and is used in parenteral nutritional agents as a glutamine supply source.

The action of alanylglutamine as a wake-up remedy has thus far been unknown.

### SUMMARY OF THE INVENTION

There is a demand for pharmaceutical products and nutritional foods, etc. created to relieve symptoms and improve the lives of people having symptoms of feeling languid after wake-up, and having a hard time awaking, etc. Specifically, an object of the present invention is to offer a wake-up remedy.

The present invention includes the following aspects.

One aspect is a wake-up remedy containing alanylglutamine or a salt thereof as an active ingredient.

Another aspect is a method of remedying difficulty in awaking characterized in that an effective amount of alanylglutamine or a salt thereof is administered to a subject in need.

Yet another aspect is a use of alanylglutamine or a salt thereof for producing a wake-up remedy.

The present invention offers a safe and effective wake-up remedy that contains alanylglutamine or a salt thereof as an active ingredient.

### DETAILED DESCRIPTION OF THE INVENTION

In the wake-up remedy of the present invention, the alanine and glutamine are the amino acids that constitute alanylglutamine. Each may be L- or D-forms, and the L-forms are preferred.

Salts of alanylglutamine include acid addition salts, metal salts, ammonium salts, organic amine addition salts, amino acid addition salts, and the like.

The acid addition salts include: inorganic acid salts such as hydrochloride, hydrosulfate, nitrate and phosphate; and organic acid salts such as acetate, maleate, fumarate, citrate, malate, lactate, α-ketoglutarate, gluconate and caprylate.

The metal salts include: alkali metal salts such as sodium salt and potassium salt; alkaline earth metal salts such as magnesium salt and calcium salt; aluminum salt; zinc salt, and the like.

Ammonium salts include salts of ammonium, tetramethylammonium, and the like.

Organic amine addition salts include salts of morpholine, piperidine, and the like.

Amino acid addition salts include salts of glycine, phenylalanine, lysine, aspartic acid, glutamic acid, and the like.

Alanylglutamine may be produced according to any method such as synthetic method, enzymatic method, or fermentation method.

Methods for producing alanylglutamine include, for example, those cited in Bulletin of the Chemical Society of Japan, 34, 739 (1961), 35, 1966 (1962), 37, 200 (1964), European Patent No. 311057, German Patent No. 3206784, Japanese Unexamined Patent Publication No. H6-234715, and WO2004/058960. Commercial products (those manufactured by Kyowa Hakko, Co., Ltd., Kokusan Kagaku, Co., Ltd., and Bachem AG, etc.) may be used for alanylglutamine.

Healthy wakefulness can be promoted by administering the wake-up remedy of the present invention to persons having subjective symptoms of feeling languid after wake-up and having a hard time awaking, etc.

Alanylglutamine or a salt thereof may be administered as it is as the wake-up remedy of the present invention, but preferably alanylglutamine is provided in any of a variety of common pharmaceutical preparations.

These pharmaceutical preparations contain alanylglutamine or a salt thereof as an active ingredient, but may also contain any other therapeutic active ingredients. Further, these pharmaceutical preparations may be produced by any method well known in the technical field of pharmaceutics by mixing active ingredients with one or more pharmaceutically acceptable carriers.

It is desirable to use the pharmaceutical preparation through a dosing route that is the most effective for the therapy, and examples thereof include oral administration and parenteral administration such as intravenous administration, intraperitoneal administration, or subcutaneous administration; but oral administration is preferred.

The dosage form may be oral preparations, such as tablets, powders, granules, pills, suspensions, emulsions, infusions/decoctions, capsules, syrups, liquid preparations, elixirs, extracts, tinctures and fluid extracts, or parenteral preparations, such as injections, drops, creams and suppositories; but oral preparations are preferred.

When preparing oral preparations, additives may be used such as excipients, binders, disintegrators, lubricants, dispersing agents, suspension agents, emulsifiers, diluents, buffers, antioxidant agents, microbial inhibitors, and the like.

Liquid preparations suitable to oral administration, for example, syrups, can be formulated by adding: water; a saccharide such as sucrose, sorbitol, or fructose; a glycol such as polyethylene glycol, or propylene glycol; an oil such as sesame oil, olive oil, or soybean oil; an antiseptic such as a p-hydroxybenzoate ester; a preservative such as a paraoxybenzoate derivative like methyl paraoxybenzoate or sodium benzoate; a flavor such as strawberry flavor or peppermint; or the like.

Further, for example, tablets, powders or granules, each of which is suitable for oral administration, can be formulated by adding: a saccharide such as lactose, sugar, glucose, sucrose, mannitol, or sorbitol; a starch such as that of potato, wheat, or corn; an inorganic substance such as calcium carbonate, calcium sulfate, sodium hydrogen carbonate, or sodium chloride; a filler such as crystalline cellulose or plant powder like licorice root powder, gentian powder, or the like; a disintegrator such as starch, agar, gelatin powder, crystalline cellulose, carmellose sodium, carmellose calcium, calcium carbonate, sodium hydrogen carbonate, or sodium alginate; a lubricant such as magnesium stearate, talc, hydrogenated plant oil, macrogol, or silicone oil; a binder such as polyvinyl alcohol, hydroxypropyl cellulose, methyl cellulose, ethyl cellulose, carmellose, gelatin, or starch paste; a surfactant such as a fatty acid ester; a plasticizer such as glycerol; or the like.

Additives generally used in foods or drinks may be added to preparations suitable for oral administration, including: sweeteners, colorants, preservatives, thickening stabilizers, antioxidant agents, coloring agents, bleaching agents, anti-fungal agents, gum bases, bitter agents, enzymes, waxes, sour agents, seasonings, emulsifiers, reinforcing agents, manufacturing agents, flavors, spice extracts, or the like.

The preparation suitable for oral administration may be used as it is or may be in such forms as a powdered food, a sheet-shaped food, a bottled food, a canned food, a retort food, a capsule food, a tablet food, a liquid food, or a drinkable preparation. Further, it may be used as a food or drink for remedying difficulty in awaking such as a health food, a functional food, a nutritional supplement food, or a food for specific health use.

Suitable parenteral administration includes, for example, an injection that preferably contains a sterilized aqueous preparation containing alanylglutamine or a salt thereof, which is isotonic to the recipient's blood. In the case of an injection, for example, a solution for injection is prepared using a carrier containing a salt solution, a glucose solution, or a mixture of a salt solution and a glucose solution, or the like.

Further, also added to these parenteral preparations may be one or more auxiliary components selected from the diluents, antiseptics, flavors, fillers disintegrators, lubricants, binders, surfactants and plasticizers described in the examples of the oral preparations, and the like.

In the wake-up remedy of the present invention, the concentration of alanylglutamine or a salt thereof is appropriately selected depending on the type of preparation, the effect expected by administration of the preparation, and the like, but, for example, the concentration in the case of an oral preparation is usually 0.1 to 100% by weight as alanylglutamine or a salt thereof, preferably 0.5 to 70% by weight, and particularly preferably 1 to 50% by weight.

The dose and the administration frequency of the wake-up remedy of the present invention may vary depending on the dosing form, the age and body weight of the patient, and the nature or the severity of the symptoms to be treated, but in general, it is administered once to several times a day usually in an amount of 5 mg to 10000 mg, preferably 50 mg to 5000 mg, more preferably 500 mg to 3000 mg per day for an adult in terms of alanylglutamine or a salt thereof. The dosing period is not particularly limited, but is usually for 1 day to 1 year, preferably 2 weeks to 3 months.

Test examples in which the wake-up remedial effect of alanylglutamine was examined are indicated below.

### Test Example 1

The results of consuming 0.5 g of alanylglutamine (manufactured by Kyowa Hakko Co., Ltd.; the same in Test Example 2) daily before going to bed continuously for 1 month in a healthy male in his 50s indicated improvement in awaking.

### Test Example 2

The results of consuming 1.5 g of alanylglutamine daily continuously for 1 month in a healthy male in his 30s indicated improvement in awaking.

Examples of the present invention are indicated below.

### Example 1

### Manufacturing of a Tablet Containing Alanylglutamine

A mixture of 136.2 kg of alanylglutamine, 36.0 kg of microcrystalline cellulose, 6.6 kg of sucrose fatty acid ester, 1.2 kg of calcium phosphate, and 20.0 kg of β-cyclodextrin are mixed using a conical blender (CB-1200 Blender, manufactured by Nihon Kansoki Co., Ltd.). The mixture obtained is compressed and molded to a wake-up remedy tablet of 250 mg with 8 mm of diameter under 10 kN of compression-molding pressure using a rotary compression molding machine (VIRGO524SS1AY, manufactured by Kikusui Seisakusho Co., Ltd.).

### Example 2

### Manufacturing of an Enteric Capsule Containing Alanylglutamine

A mixture of 20 kg of the mixture produced in Example 1 and 0.2 kg of silicon dioxide are mixed and agitated. The mixture obtained is put into a capsule-filling machine to fill 20,000 tablets of gelatin Number 2 hard-capsules, and hard-capsules are obtained. The surfaces of the hard-capsules are coated with a zein solution using a High Coater HCT-48 (manufactured by Freund Corporation) to produce 20,000 wake-up remedy enteric capsules.

### Example 3

### Manufacturing of an Enteric Tablet Containing Alanylglutamine

The surfaces of the tablets produced in Example 1 are coated with a shellac solution using a High Coater HCT-48 (manufactured by Freund Corporation) to produce wake-up remedy enteric tablets.

### Example 4

### Manufacturing of a Beverage Containing Alanylglutamine

An amount of 1.28 kg of alanylglutamine, 3 kg of erythritol, 0.05 kg of citric acid, 3 g of artificial sweetener, and 0.06 kg of flavor are stirred and dissolved in 50 L of water at a temperature of 70° C. After the pH of the solution is adjusted to 3.3 with citric acid, the solution is sterilized using plate sterilization and filled into bottles. The bottles are sterilized using a pasteurizer, and thus a wake-up remedy drink is produced.

According to the present invention, a wake-up remedy containing alanylglutamine or a salt thereof as an active ingredient can be provided.

1. A wake-up remedy comprising alanylglutamine or a salt thereof as an active ingredient.

2. A method of remedying difficulty in awaking, comprising the step of administering an effective amount of alanylglutamine or a salt thereof to a subject in need.

3. A use of alanylglutamine or a salt thereof for producing a wake-up remedy.

4. The method of remedying difficulty in awaking according to claim 2, wherein the alanylglutamine or salt thereof is administered in an amount of 5 mg or more and 10, 000 mg or less per day.

5. The method of remedying difficulty in awaking according to claim 2, wherein the alanylglutamine or salt thereof is administered in an amount of 50 mg or more and 5, 000 mg or less per day.

6. The method of remedying difficulty in awaking according to claim 2, wherein the alanylglutamine or salt thereof is administered in an amount of 500 mg or more and 3, 000 mg or less per day.

7. The method of remedying difficulty in awaking according to claim 2, wherein the alanylglutamine or salt thereof is administered for a period of 1 day or more and 1 year or less.

8. The method of remedying difficulty in awaking according to claim 2, wherein the alanylglutamine or salt thereof is administered for a period of 2 weeks or more and 3 months or less.

9. The method of remedying difficulty in awaking according to claim 2, further comprising the step of producing a sterilized aqueous preparation comprising the alanylglutamine or salt thereof, which is isotonic to the subject's blood,
wherein the administration is parenteral.

10. The method of remedying difficulty in awaking according to claim 2, further comprising the step of producing a preparation comprising the alanylglutamine or salt thereof at a concentration of 0.1% or more by weight,
wherein the administration is oral.

11. The method of remedying difficulty in awaking according to claim 2, further comprising the step of producing a preparation comprising the alanylglutamine or salt thereof at a concentration of 0.5% or more and 70% or less by weight,
wherein the administration is oral.

12. The method of remedying difficulty in awaking according to claim 2, further comprising the step of producing a preparation comprising the alanylglutamine or salt thereof at a concentration of 1% or more and 50% or less by weight,
wherein the administration is oral.
